(19) United States
(12) Patent Application Publication
(10) Pub. No.: US 2009/0221956 A1
(43) Pub. Date: Sep. 3, 2009

(54) MULTI-PARAMETER MONITORING DEVICE FOR USE WITH CENTRAL AND INTRAVENOUS ADMINISTRATION OF MEDICATION

(75) Inventors: Daniel J. Abrams, Denver, CO (US); Raymond Bunch, Phoenix, AZ (US); Michael Royals, Pierce, CO (US)

Correspondence Address: MORRISON & FOERSTER LLP, 755 PAGE MILL RD, PALO ALTO, CA, 94304-1018, US

(73) Assignee: The Regents of the University of Colorado, Denver, CO (US)

(21) Appl. No.: 12/161,302

(22) PCT Filed: Jan. 22, 2007
(86) PCT No.: PCT/US07/01692

§ 371 Date: Dec. 3, 2008

Related U.S. Application Data
(60) Provisional application No. 60/760,813, filed on Jan. 20, 2006.

(51) Int. Cl. A61M 5/142

(52) U.S. Cl. 604/66

(57) ABSTRACT

The invention generally relates to a multi-parameter monitoring device for in-line use to monitor a therapeutic agent solution passing therethrough prior to being centrally administered in connection with treatment of a CNS-related condition or disorder, e.g., a neuro-psychiatric disorder. In another aspect, the invention relates to a multi-parameter monitoring device for in-line use to monitor a therapeutic agent solution passing therethrough prior to being (a) intravenously administered to a patient in connection with a treatment, such as is the case where the therapeutic agent solution comprises an IV administerable solution, or (b) administered to a patient in connection with a treatment for diabetes, such as is the case where the therapeutic agent comprises insulin.

### RELATED APPLICATIONS

The present application claims the benefit of the filing date of U.S. provisional application No. 60/760,813, entitled, “New Drug Delivery Technique and Multi-parameter Monitoring Device for Use with Subarachnoid Release of Intrathecal Medication”, filed on Jan. 21, 2006, the entire contents of which are specifically hereby incorporated by reference for all purposes.

### FIELD OF THE INVENTION

The present invention relates generally to systems, techniques and associated devices for administering medication via central administration route, as well as intravenously (IV) administration route.

### BACKGROUND OF THE INVENTION

### Central Administration

Lumbar continuous intrathecal treatment has been used routinely and frequently for more than 10 years. Patients in the US have had this mode of therapy for pain, spasticity, and to a very limited extent, for neoplasia, medtronic.com/neuro/paintherapies/pain_treatment. Integrated catheter and computerized pump delivery systems are commercially available through several vendors, and several new microinjection systems are in development. On an individual case basis, single- or multiple-dose intrathecal cranial injections have been used to treat CNS infections by neurosurgeons injecting antifungals and antibacterials with Ommaya reservoirs and intraventricular catheters in a saline or equivalent carrier, at neutral pH.

In a series of experiment from the 1970s, medications were administered ICV and in the spinal axis. Small molecules (amino acids, chemotherapeutic agents and nucleic acid analogs) were injected ICV and pain medications were injected into the spine. A primary finding from those studies is that the degree of hydrophobicity in a compound's structure predicted bio-distribution (amount distributed and rate of distribution) of opiate active medications into the central nervous system parenchyma when medications are administered directly into the CSF. Subsequently, methods attempting to quantify how fast and how far the pain medications penetrated into the brain have been developed. There is limited data in humans related to ICV administered medications for psychiatric disease in terms of how these medications permeate into the brain, and at what rate. Clinical experience with other medications has been limited to intermittent single bolus injection primarily for infection.

Known intrathecally administered pain management systems designed for treating human spasticity and pain currently use an intrathecal intraspinal catheter assembly and an implantable pump. By way of example, a programmable infusion system manufactured and distributed under the brand name SynchroMed® EL for delivering pain management therapies, directly into the central nervous system (CNS) of humans, is marketed by Medtronic, see, e.g., medtronic.com/paintherapies/pain_treatment. FIGS. 1A-1B (PRIOR ART) and FIG. 2 (PRIOR ART) illustrate the SyncroMed® pump.

A pump having functionality similar to that depicted in FIGS. 1A-1C is described in U.S. Pat. No. 6,360,784 issued for an “implantable drug infusion pump” (IDIP) used for administering pain killers, nerve growth factor, and anti-spasticity drugs to the intrathecal region of the spinal column (i.e., intrathecal delivery). The figure labeled FIG. 6 in U.S. Pat. No. 6,360,784 is likewise incorporated herein and labeled FIG. 3 (PRIOR ART), for purposes of illustrating one example of how a therapeutic agent may be filled into an implanted pump (IDIP 18). Columns 1 and 2 of U.S. Pat. No. 6,360,784 are also fully incorporated by reference herein for the technological detail shown in FIG. 3 (PRIOR ART). One can appreciate that the medication is injected by way of a hypodermic needle attached to a plunger syringe having a volume reserve sized to house a sufficient amount of the medication.

U.S. Pat. No. 6,656,172 describes a method for treating severe tinnitus employing implanting a catheter into a patient and administering an associated therapeutic agent intrathecally into the patient's cerebrospinal fluid. FIG. 5 of U.S. Pat. No. 6,656,172 is a cross-section of the neck and head regions of a human body, illustrating placement of the catheter 38 described in that patent for use to treat tinnitus.

Currently, medications used for long term spinal intrathecal drug delivery include fentanyl, sufentanil, meperidine, morphine, baclofen, ziconitide, clonidine, and bupivacaine. Others, including gabapentin and BDNF, currently remain under investigation. These medications are water soluble, presented at a neutral pH and are mixed in isotonic buffers without buffers or solubilizing agents. There are no drugs specifically approved for ICV use, although chemotherapeutics (including cytarabine and methotrexate) and antimicrobials (including amphotericin B) have been used intermittently.

### The Blood-Brain Barrier

The blood-brain barrier (BBB) is a gate that controls the influx and efflux of a wide variety of substances and consequently restricts the delivery of drugs into the central nervous system (CNS). Inadequate drug delivery is a major factor that explains the poor responses to CNS drugs (i.e. antipsychotic). Various strategies have been devised/attempted to circumvent the BBB in order to increase drug delivery to the CNS. Neurotoxicity is a big concern with increased penetration of drugs into the CNS and systemic toxicity remains the limiting factor for most methods that use intravascular delivery.

The bulk of the brain and the spinal cord are surrounded by a specially secreted clear fluid called the cerebrospinal fluid (CSF). Chemical substances such as metabolites move relatively freely from the alimentary canal into the blood, but not into the CSF. As a result, the blood levels of sugars, amino acids or fatty acids fluctuate over wide range while their concentrations in the CSF remain relatively stable. The same is true for hormones, antibodies, certain electrolytes, and a variety of drugs. Injected directly into the blood they act rapidly on peripheral tissues such as the muscles, heart, or glands but they have little or no effect on the central nervous system (CNS). When administered into the CSF, however, the same substances exert a prompt and strong action. Once substances have found their way into the CSF, they can readily diffuse into the tissues of the brain. The entry of hydrophilic and relatively large molecules into the CNS is restricted by the existence of a BBB.

### Computerized Devices, Integrated Circuits (ICs), Memory & Storage.

I. Digital computers. A processor is the set of logic devices/circuitry that responds to and processes instructions to drive a computerized device. The central processing unit (CPU) is considered the computing part of a digital or other type of computerized system.

Often referred to simply as a processor, a CPU is made up of the control unit, program sequencer, and an arithmetic logic unit (ALU)—a high-speed circuit that does calculating and comparing. Numbers are transferred from memory into the ALU for calculation, and the results are sent back into memory. Alphanumeric data is sent from memory into the ALU for comparing. The CPUs of a computer may be contained on a single ‘chip’, often referred to as microprocessors because of their tiny physical size. As is well known, the basic elements of a simple computer include a CPU, clock and main memory; whereas a complete computer system requires the addition of control units, input, output and storage devices, as well as an operating system. The tiny devices referred to as ‘microprocessors’ typically contain the processing components of a CPU as integrated circuitry, along with associated bus interface. A microcontroller typically incorporates one or more microprocessor, memory, and I/O circuits as an integrated circuit (IC). Computer instruction(s) are used to trigger computations carried out by the CPU. Frequency counters are digital indicating meters for measurement and display of input signals in the form of square wave(s) and pulse(s). Binary counters are digital circuits that have a clock input and one or more count output; the count output may give the number of clock cycles for a clock input, or may be employed to count pulses for an input digital waveform.

II. Microelectronics—Structures and Devices. Microelectronics is that area of electronics technology associated with the fabrication of electronic systems or subsystems using extremely small (microcircuit-level) components. Semiconductor fabrication and processing is driven by the computer-electronics industry. The demands for greater capability and faster data collection and processing of smaller-sized computerized units result in a demand for smaller-and-smaller integrated circuit (IC) microcircuits. “Chip” and/or ‘microchip’ are often used to refer to any one or interrelated operational set of micro-miniaturized, electronic circuits, or microdevices—including microprocessors—that have been designed for use as electrical components, processors, computer memory, as well as countless special purpose uses in connection with consumer goods and industrial products; larger sized similarly-styled structures on the order of 1 cm and up, may also be referred to as ‘chip’. The terms chip, integrated circuit (IC), and microchip are often used interchangeably within the electronics industry: the smaller microchips can hold a handful to hundreds-of-thousands of transistor/electrical devices (tiny chips of around 1/16″ square by 1/30″ thick); whereas larger-sized microchips sized on the order of ½inch², are capable of containing many millions of transistor/electrical devices.

III. Computer Memory and Computer Readable Storage. While the word ‘memory’ has historically referred to that which is stored temporarily, with storage traditionally used to refer to a semi-permanent or permanent holding place for digital data—such as that entered by a user for holding long term—more-recently, the definitions of these terms have blurred. A non-exhaustive listing of well known computer readable storage device technologies are categorized here for reference: (1) magnetic tape technologies; (2) magnetic disk technologies include floppy disk/diskettes, fixed hard disks (often in desktops, laptops, workstations, etc.), (3) solid-state disk (SSD) technology including DRAM and ‘flash memory’; and (4) optical disk technology, including magneto-optical disks, PD, CD-ROM, CD-R, CD-RW, DVD-ROM, DVD-R, DVD-RAM, WORM, OROM, holographic, solid state optical disk technology, and so on.

### Historical Perspective: Treating Schizophrenia.

Schizophrenia is a disabling illness frequently ineffectively treated using available modalities. Ineffective treatment of schizophrenia occurs as a result of significant drawbacks of commercially-available antipsychotics: These include medication side effects, failure to achieve therapeutic doses, and overall patient compliance. Prospective studies indicate that 50-70% of schizophrenia patients have a persistent and chronic course.

Estimates of the overall cost of schizophrenia in the human population is huge, ranging in the tens-of-billions of dollars in the United States, alone. Currently-available oral and intramuscular treatment modalities have limited ability to overcome the efficacy problems of current pharmacologic therapies because of significant systemic side effects, among other limitations. Conventional formulations of the ‘newer’ atypical antipsychotics and older typical antipsychotics, are currently administered in oral and long acting intramuscular (IM) forms. Clozapine is often used as an oral atypical antipsychotic medication, in the treatment of refractory schizophrenia. However, patients taking current formulations of clozapine face risk side effects including sleepiness, weight gain and lowered blood pressure, as well as life-threatening systemic toxic side effects (such as myocarditis and agranulocytosis).

U.S. Pat. No. 5,975,085 describes a technique for using a drug and/or electrical stimulation for treating schizophrenia by means of an implantable signal generator and electrode and an implantable pump and catheter; the catheter and electrodes having been surgically implanted directly into the brain to infuse the drugs and provide the electrical stimulation. The technique described and shown in '085 is extremely invasive—as one can appreciate—since the catheter and electrodes are implanted deep into the brain. Applicants know of no attempt to employ this invasive technique to treat schizophrenia in a human patient. Columns 1 and 2 of U.S. Pat. No. 5,975,085 are incorporated by reference herein for its general background discussion and information concerning the disease of schizophrenia.

### Historical Perspective: Limitations of Conventional/Current Treatments for Schizophrenia.

Both typical and atypical antipsychotics have multiple significant side effects including movement disorders, hypotension (typicals) and diabetes (atypicals). Other significant problems besides side effects include extremely poor compliance with oral medications. Intramuscular formulations, (including Resperidone and Olanzapine for the atypicals, and haloperidol in the typicals), are limited by the inability to halt medication once it is injected, “constant dosing”, and retention of a significant systemic side effect profile. Transdermal systems under development may improve compliance, eliminate the pain of an intramuscular injection, and potentially can be discontinued abruptly but they still have the limitations of constant dosing and significant side effects. Side effects remain a profound issue in antipsychotic administration using conventional methods, and can result in patient death (e.g., bone marrow failure with clozapine) and patient illness (e.g., liver toxicity and cardiac conduction deficits). Clozapine has been found to be superior in treatment of disabling the negative symptoms associated with schizophrenia (disorganization, cognitive dulling and socialization).

Current therapies for treating chronic maladies, such as diabetes, and for delivering therapeutic solutions intravenously for a variety of reasons, whether short or long term drug delivery, replacement of lost blood and other fluids, electrolyte replacement, and so on, are often lacking adequate monitoring and control of delivery of the therapeutic solution. Currently, there is no retrofit-adaptable, multi-parameter monitoring device such as that contemplated herein, for automatically monitoring solutions as they pass from the IV bag unit, a pump unit, or other such receptacle, through a catheter assembly and into the blood stream.

### SUMMARY OF THE INVENTION

To address such needs and others, provided herein is a multi-parameter monitoring device for use in connection with the central or intravenous administration of medications.

In one aspect, the invention relates to a multi-parameter monitoring device for inline use to monitor a therapeutic agent solution passing therethrough prior to being centrally administered in connection with treatment of a CNS-related condition or disorder, e.g., a neuro-psychiatric disorder. In another aspect, the invention relates to a multi-parameter monitoring device for in-line use to monitor a therapeutic agent solution passing therethrough prior to being (a) intravenously administered to a patient in connection with a treatment, such as is the case where the therapeutic agent solution comprises an IV administerable solution, or (b) administered to a patient in connection with a treatment for diabetes, such as is the case where the therapeutic agent comprises insulin.

In certain aspects of the invention, the new device includes: (a) a fluid channel through which the therapeutic agent solution is directed after having exited an automatically-driven pump mechanism, or in the case of IV fluid, an IV receptacle; (b) an integrated circuit (IC) unit comprising at least one parameter-detection element adapted for collecting information concerning a parameter of the therapeutic agent solution while the solution flows through the fluid channel; and (c) on-board the integrated circuit (IC) unit is a processor adapted for processing at least a portion of any said information so collected.

These and other aspects of the invention will become apparent to one of skill in the art.

### BRIEF DESCRIPTION OF THE DRAWINGS

For purposes of illustrating the innovative nature plus the flexibility of design and versatility of the new device and associated technique for administering (e.g., via intrathecal delivery) a therapeutic agent to a subject, the following figures are included.

One can readily appreciate the advantages as well as novel features that distinguish the instant invention from conventional devices and techniques. The figures are intended by way of example, only, and are not intended to limit the disclosure hereof.

FIGS. 1A-1B are isometric sketches, and FIG. 1C is a digital photo, representing a pump unit 10, 20 having an exit port from which a catheter connection assembly extends (comprised of 32, 33, 30 as shown in FIG. 1C) to interconnect an intrathecal catheter length 34. As is well known, pump apparatuses 10, 20 are typically implanted within a patient's body.

FIG. 2 is a digital photo of another (isometric-type) view of components 32, 33, 30.

FIG. 3 is a schematic-isometric illustrating an example of a currently available method for filing a medicine access port, such as that represented at 98 in FIG. 9, of a pump apparatus/unit (e.g., those at 10, 20). Applying pressure to plunger 30 causes medicine filled within a reservoir of pharmacy syringe 12 to pass through filter 14, filling tube 16, needle 28, and into IDIP 18 via the access port.

FIG. 4 is a high-level schematic, not to scale, depicting the location of intrathecal delivery of a therapeutic agent/injectable biomaterial into cerebrospinal fluid within the subarachnoid/intrathecal space, by way of definition.

FIG. 5 is a high-level schematic, not to scale, depicting a monitoring device 100 according to the invention.

FIG. 6 is a high-level schematic, not to scale, depicting detection-functionalities of an IC (‘integrated circuit’) chip-style micro-device/unit 110.

FIG. 7 is a high-level schematic, not to scale, depicting positional relationship of a pump unit 10A, monitoring device 100A, intrathecal catheter assembly within a patient body 200A.

FIG. 8 is a high-level schematic, not to scale, depicting positional relationship of a pump unit 10B, monitoring device 100B, intrathecal catheter assembly within a patient body 200B.

FIG. 9 is a high-level schematic, not to scale, depicting positional relationship of an IV bag unit 312A, monitoring device 320A, and IV catheter assembly in communication with the blood stream within a patient body 300A.

FIG. 10 is a high-level schematic, not to scale, depicting positional relationship of an insulin pump unit 312B, monitoring device 320B, and IV catheter assembly in communication with the blood stream within a patient body 300B.

### DETAILED DESCRIPTION OF THE INVENTION

Current therapies for treating CNS-related conditions and disorders such as schizophrenia and other such psychiatric disorders (e.g., seizures) are only available for per oral (PO) or intravenous (IV) administration. In order for a drug give PO or IV to cross the BBB—and reach the site of action, the CNS, in therapeutic concentrations—it is currently necessary to administer the drug in high doses, thus, increasing severity of unwanted side effects.

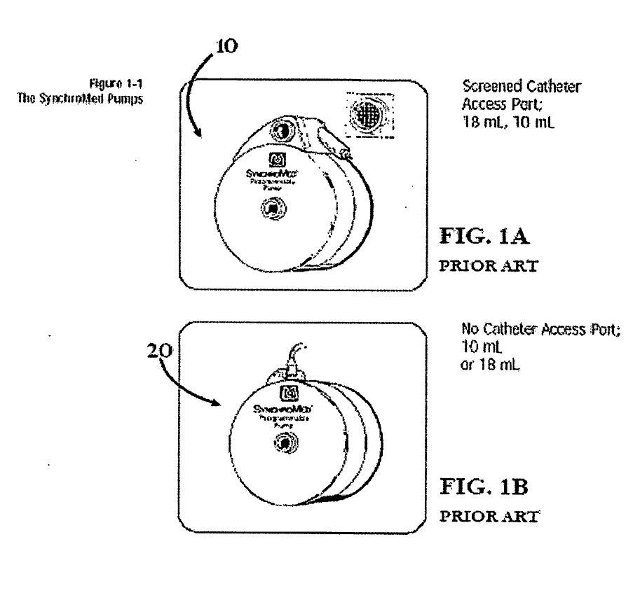

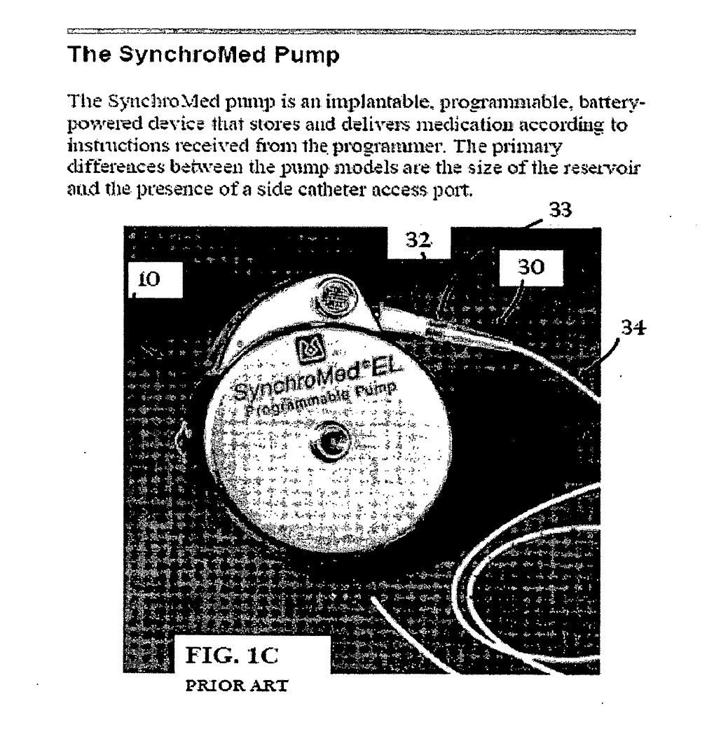

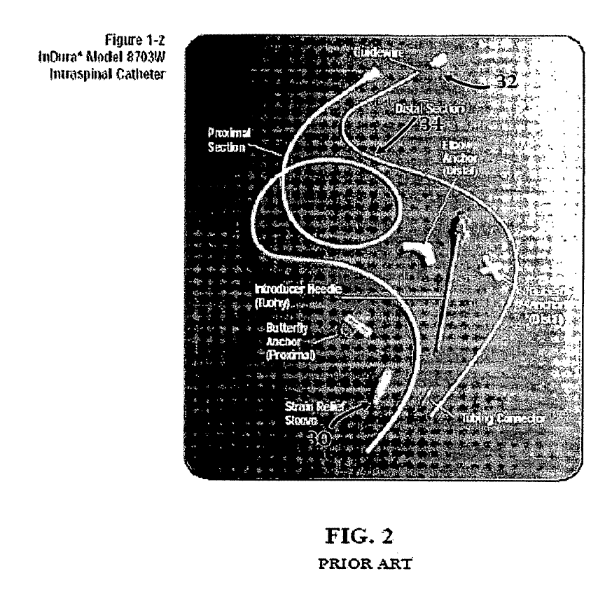

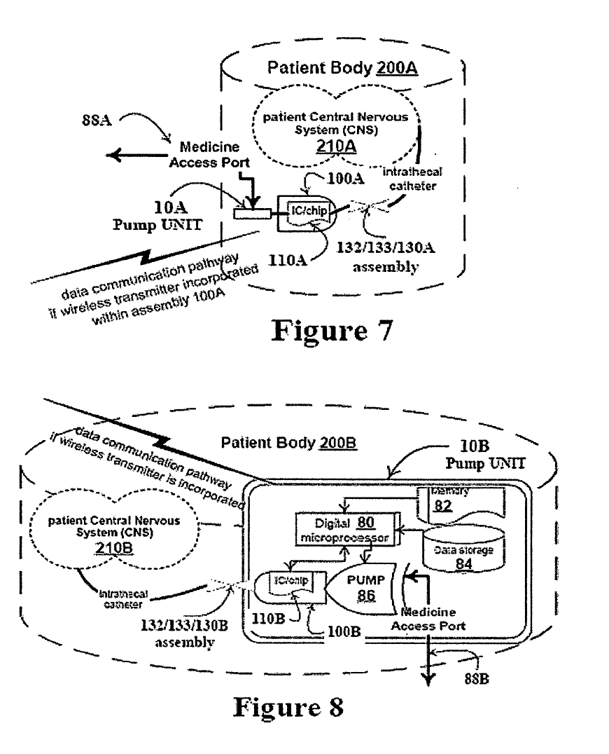

A new multi-parameter monitoring (‘MPM’) device and technique for use in connection with the central administration of medication for the treatment of subjects suffering from, e.g., neuro-psychiatric diseases, such as schizophrenia and other CNS-related conditions and disorders (e.g., bipolar mood disorder, severe depression, shizoaffective disorder, etc.), is described herein. While the MPM device and technique, by way of example, will be employed to administer a reformulation of the antipsychotic, clozapine, it is not limited thereto. But rather, the MPM device of the invention—whether incorporated as a retrofit component for in-line use with a catheter assembly (such as that depicted in FIGS. 1C and 2) and pump (such as that depicted at 10 in FIGS. 1A-1C and FIG. 7 at 10A), or designed as an in-line feature incorporated within a new unitary pump assembly (i.e., integrated with a catheter assembly) (such as that depicted in FIG. 8 at 10B)—may be adapted for many types of therapeutic agents to monitor central treatments targeted for a variety of different psychiatric and neurological disorders/neuro-psychiatric diseases, as well as intravenous treatments for a variety of indications, such as diabetes.

By viewing the figures and associated representative structure embodiments, one can farther appreciate the unique nature of core as well as additional and alternative features of the new device and associated technique for central delivery of a therapeutic agent to a subject, as well as alternative features of the MPM device employed for intravenous IV-type delivery to a subject.

The term subject, as used throughout and traditionally contemplated, include members of the animal kingdom, humans and non-human animals (livestock, pets, wild game, domestically maintained ocean mammals, and so on) to the extent central delivery is plausible for the aspect of the invention directed to central delivery of therapeutic solutions, and to the extent for which IV and insulin delivery is adaptable for the aspect of the invention directed to delivery of therapeutic solutions into the blood stream. Reference will be made to various features—especially as depicted in the high-level schematics labeled FIG. 7-10—by way of back-and-forth reference and association to respective figures.

By way of background, FIGS. 1A-1B (PRIOR ART) are isometric sketches, and FIG. 1C (PRIOR ART) is a digital photo, representing a pump unit 10, 20 having an exit port from which a catheter connection assembly extends (comprised of 32, 33, 30 as shown in FIG. 1C) (PRIOR ART) to interconnect an intrathecal catheter length 34. As is well known, pump apparatuses 10, 20,18 are typically implanted within a patient's body. FIG. 2 (PRIOR ART) is a digital photo of another (isometric-type) view of components 32, 33, 30.

In the context of the present invention, one aspect is directed to a multi-parameter monitoring (MPM) device for automatic monitoring, and associated new technique, for use in connection with the central administration, e.g., intrathecal administration, of medication in the treatment of patients suffering from, e.g., CNS-related conditions or disorders including neuro-psychiatric diseases, such as schizophrenia.

In one embodiment, the MPM device is for use in-line with a central administration device to monitor a therapeutic agent solution passing therethrough prior to being centrally administered in connection with the treatment of a CNS-related condition or disorder. The MPM device may generally include, as described in further detail herein, (a) a fluid channel through which the therapeutic agent solution is directed after having exited an automatically-driven pump mechanism; (b) an integrated circuit (IC) unit comprising at least one parameter-detection element adapted for collecting information concerning a parameter of the therapeutic agent solution while the solution flows through said fluid channel; and (c) on-board said integrated circuit (IC) unit is a processor adapted for processing at least a portion of any said information so collected. In certain embodiments, the information may then be used so as to better control the dosage and treatment regimen of the subject, either automatically or manually through physician interaction.

The MPM device may be releasably-connectable, such as when used in a ‘stand alone retrofit’ manner, in-line within an intrathecal or central administration pump catheter assembly such as that depicted in FIG. 2 (although the invention is not so limited), comprising a length of catheter tubing (e.g., item 34, FIG. 1C) and a port-connector (e.g., item 32, FIG. 1C). Alternatively, the MPM device may be integrated with the central administration pump catheter assembly. The port-connector may be adapted for connection of the catheter assembly to an exit port of an implantable unit (e.g. items 10, 10A, 10B as labeled) that comprises the automatically-driven pump mechanism. The device may be generally cylindrical in shape and further adapted for threaded engagement with the port-connector of the catheter assembly and for engagement with the implantable unit's exit port. Further, both the automatically-driven pump mechanism, in communication with the device, may be built as contained within a housing (e.g., 10B, FIG. 8) for an implantable unit; the device also being in communication with an exit port of the implantable unit. Here, the therapeutic agent solution flows through the fluid channel prior to exiting the implantable unit through its exit port and into a port-connector (e.g., may be structured similar to item 32, FIG. 1C) of an intrathecal catheter assembly having a length of catheter tubing (e.g., item 34, FIG. 1C).

Parameters of interest detected/measured by the MPM device, may include, but are not limited to: speed of flow, pressure, temperature, density, and so on, of the therapeutic agent solution while flowing through the device; concentration of constituents of the solution (to monitor and control dosage as well as administer medication in a continuous fashion); concentration of components added, downstream, to an original solution being stored in an automatic pump unit; contaminant detection (presence as well as amount/concentration); precipitation quantity to optically detect particles larger than, or of a, pre-selected size that precipitate out of the solution; pH of therapeutic agent flowing into catheter assembly (maintain a tolerable/physiological range as necessary for intrathecal delivery downstream); and so on.

More particularly, in other embodiments, parameters of interest may include: speed of flow of the therapeutic agent solution through the fluid channel (note, ‘speed’ is intended to include that situation where flow stops, i.e., where velocity=0); pressure within the fluid channel while the therapeutic agent solution is passing therethrough; density of the therapeutic agent solution while passing through the fluid channel; temperature within the fluid channel while the therapeutic agent solution is passing therethrough; concentration of a selected constituent of the therapeutic agent solution (such as a drug or medicine, and/or any other constituent of the therapeutic agent solution which will flow though the catheter); concentration of a component added to the therapeutic agent solution (note, ‘component added’ is intended to include anything that might be added to an original solution formulation injected into the pump, for example, the formulation is too weak/degraded due to sitting in the pump/IV bag for too long, so more of an initial constituent ‘x’ has to be added, and includes a component added well after a pump has been implanted, for example, as a result of a new prescription written for the patient, or a component to a constituent that has a short shelf-life, so the addition is made the day the constituent is mixed and cannot be stored, and so on); presence of a contaminant;

concentration of a contaminant (note, in any case, ‘contaminant’ is intended to include anything that is unwanted, whether toxic or a benign impurity); precipitation quantity of a particle having precipitated out of the therapeutic agent solution (to include detecting precipitation of ‘x’ as an indicator of changes in the composition of the solution, and/or detecting ‘x’ in connection with an infection, and so on); pH of the therapeutic agent solution; tonicity of the therapeutic agent solution; and conductance of the therapeutic agent solution.

In a particular embodiment, the MPM device may be used to monitor for infection and/or blockage of the central administration device, e.g., by monitoring saliity, pH, particulate matte, flow, pressure, etc. of the therapeutic agent solution.

In certain embodiments, the MPM device can include an alarm in communication with the processor. This alarm is preferably set to activate (e.g., to sound, begin vibrating, and so on) from within when any of the information so collected indicates a variety of conditions, such as a condition occurring outside a physiologic (i.e., physically-tolerable) or predetermined range, for example, or set to activate to indicate some other condition of concern or of interest (such as the therapeutic agent solution has stopped flowing, is flowing too fast, or has not been dosed in a selected manner, and so on). In certain optional embodiments, the MPM device may include an automatic shutoff that may be triggered, e.g., by the alarm to shut the central administration device off if certain predetermined conditions are detected. Alternatively, the automatic shutoff may be triggered independently of the alarm.

The integrated circuit (IC) unit preferably further includes a second parameter-detection element adapted for collecting second information concerning a second parameter of the therapeutic agent solution while the solution flows through the fluid channel, the processor further adapted for processing at least a portion of any of the second information so collected. Additionally, the integrated circuit (IC) unit can further include: (a) a third parameter-detection element adapted for collecting third information concerning a third parameter of the therapeutic agent solution while the solution flows through the fluid channel; (b) a fourth parameter-detection element adapted for collecting fourth information concerning a fourth parameter of the therapeutic agent solution while the solution flows through the fluid channel; and so on to gather information of a multitude of a wide variety of parameters of the solution; the processor, likewise, being further adapted for processing at least a portion of any of the third information and a portion of any of the fourth information so collected.

In other embodiments, the MPM device may include a module configured to record and/or report collected data. For example, the module may report data at predetermined intervals to a physician or download data at predetermined intervals to a computer for analysis and monitoring. Any suitable communication mechanism may be used, e.g., telemetry, wireless, etc.

In one particularly embodiment, in the case of schizophrenia, an antipsychotic known by its generic name as clozapine, is available in dosages and formulations taken by human patients, orally. The Novartis Corporation manufactures and distributes the drug clozapine under the brand name Clozaril®. Generic forms of clozapine are marketed by companies such as Zenith Goldline and Mylan Pharmaceuticals. Clozaril® is an atypical antipsychotic medication for patients with schizophrenia. Intrathecal delivery of clozapine represents a radical shift in psychiatric or neurological treatment, since intrathecal use of psychiatric agents has not been put into practice. In another aspect, the invention is directed to a new device and technique for automatic monitoring of IV solutions containing therapeutic agents used in IV delivery as well as therapeutic solutions containing the hormone insulin for diabetic patients.

### A. Definitions

Catheter: a thin flexible tube made of a flexible material, such as rubber or a plastic, used to insert or remove fluids from the body.

Cerebrospinal/cerebral spinal fluid (CSF): a clear fluid produced by the choroid plexus in the ventricles of the brain that bathes the brain and spinal cord giving them support and buoyancy to protect from injury.

Intrathecal space: the space surrounding the spinal cord through which CSF flows; also called the subarachnoid space.

Intrathecal preparations deliver drugs into the cerebrospinal fluid within the subarachnoid space.

Central nervous system (CNS) drugs encompass several major therapeutic classes including antidepressants, anxiolytics, mood stabilizers and antipsychotics. Antipsychotic medications (or simply, antipsychotics) are potent psychotropic drugs used primarily in the treatment of psychotic disorders such as schizophrenia.

### B. Exemplary Therapeutic Agents and Uses

The therapeutic agent solution can comprise a formulation of clozapine (for treatment of schizophrenia); or the agent can comprise one or more medicine listed below, among others: immunoglobulins, tegretol, lithium, felbamate, phenytoin, lamictal, phenobarbital, olanzapine, risperidone, ethosuximide, L-Dopa, parnate, phenelzine, isocarboxazid, clomipramine, bromocriptine, clozapine, progabide, oxcarbamazipine, clorazepate, etobarb, ziprasidone, seroquel, aripiprazole, zonisamide, methadone, buprinorphine, duramorph, clonidine, clonazapate, diazepam, temezapam, oxazepam, lorezapam, luvox, paroxetine, fluoxetine, amitryptiline, nortryptiline, desipramine, amantadine, salicylic acid, ibuprofen, acetimonophen, haloperidol, loxitane, navane, mellaril, thorazine, moban, trilafon, stelazine, prolixin, prednisilone, dexamethasone, carbamezapine, valproic acid, clonezepam, ethosuximide, oxezapam, alprazolam, bromazepam, chlordiazepoxide, clobazam, clonazepam, estazolam,, flurazepam halazepam, ketazolam, quazepam, prazepam, temazepam, triazolam, nitrazepam, diamox, ACTH, carbatrol, diastat, felbamate, valproic acid, carbamezapine, lorazepam, flurazepam, clonazepam, triazolam, chlordiazepoxide, temazepam, alprazolam, sulfasalazine, acetaminophen, cafergot, and naloxone, chlorpromazine, fluphenazine, loxapine, thioridazine, thiothixine, prochlorperazine, trifluoperazine, methylprestone, lorazepam, flurazepam, clonazepam, triazolam, chlordiazepoxide, temazepam, alprazolam, hydroxyzine oxcarbazepine, zarontin, lamotrigine, aripriprazole, and olanzapine.

The CNS-related conditions or disorders that may be treated using the MPM-device of the present invention include any known CNS-related condition or disorder, e.g., neuro-psychiatric disorders such as schizophrenia; bipolar mood disorder; depression;

shizoaffective disorder; Dementia/Alzheimer's disease; Epilepsy; Encephalitis; Multiple sclerosis; Closed head injury; Anxiety; Psychosis; Parkinson's disease; Drug addiction, etc.

### C. Exemplary Embodiments:

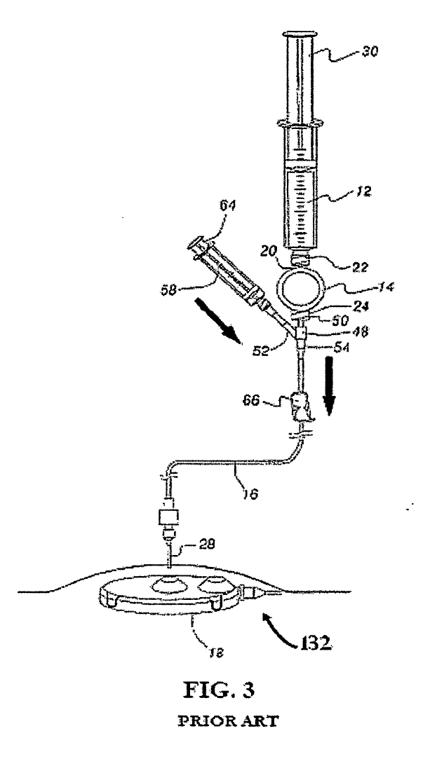

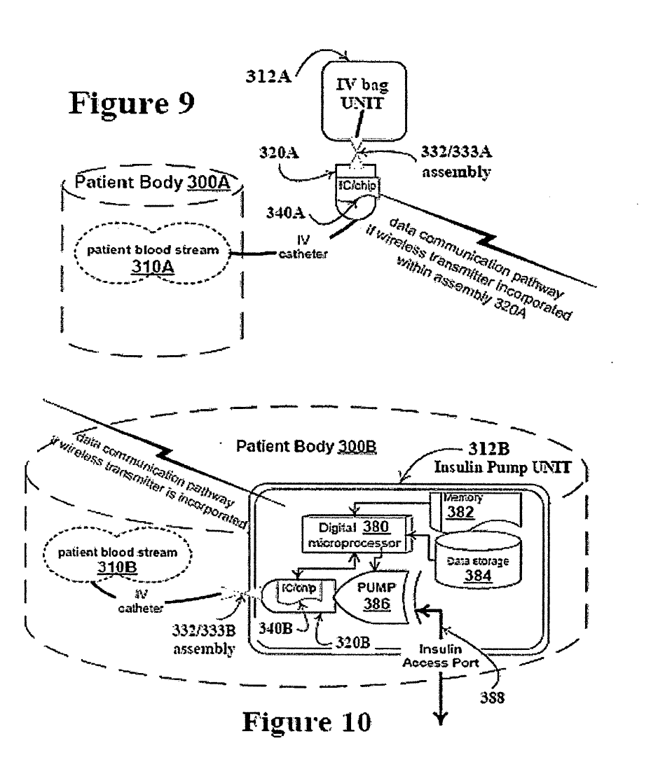

FIG. 3 is a schematic-isometric illustrating an example of a currently available method for filing a medicine access port, such as that represented at 98 in FIG. 9, of a pump apparatus/unit (e.g., those at 10, 20). Applying pressure to plunger 30 causes medicine filled within a reservoir of pharmacy syringe 12 to pass through filter 14, filling tube 16, needle 28, and into IDIP 18 via the access port.

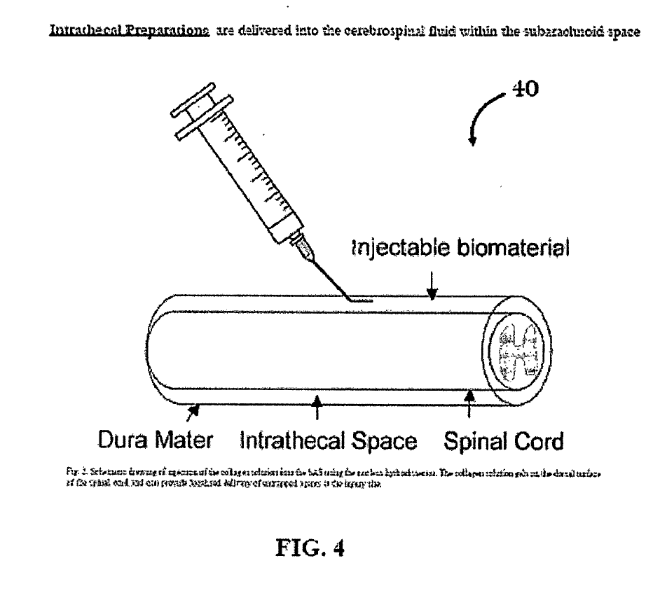

The programmable pump unit alternatives labeled 10 and 20 in FIGS. 1A-1C and 10 at 18 in FIG. 3 each have an implantable housing in which the pump mechanism and associated processor for controlling the automatic pump reside. As labeled (FIGS. 1C and 2) a length of catheter 34 is connected to pump unit 10, 20, through an assembly of suitable components (a metal guide extender 33 disposed between a clear strain relief sleeve 30 and a coupling piece 32). The pump unit 10, 20 is surgically implanted and the distal end of the catheter placed in communication with the intrathecal space (e.g., see FIG. 4) for intrathecal delivery of the solution that has been filled into the pump unit. For further reference, see FIG. 3 at 52/58/64 and at 12/30, as well as the associated description of filling a pump unit (such as that at 18, FIG. 3), set forth in U.S. Pat. No. 6,360,784 from which FIG. 3 originated, by way of example. FIG. 4 is a high-level schematic, not to scale, depicting the location of intrathecal delivery of a therapeutic agent/injectable biomaterial into cerebrospinal fluid within the subarachnoid/intrathecal space, by way of definition. The distal end of the catheter may be equipped with a flattened head with holes—for example over ˜5 cm—for infusion of medication into the CSF space. Surgical lumbar insertion of an implant catheter via the subarachnoid space, permits transport of the therapeutic solution to the cerebellum and possibly over the cortex.

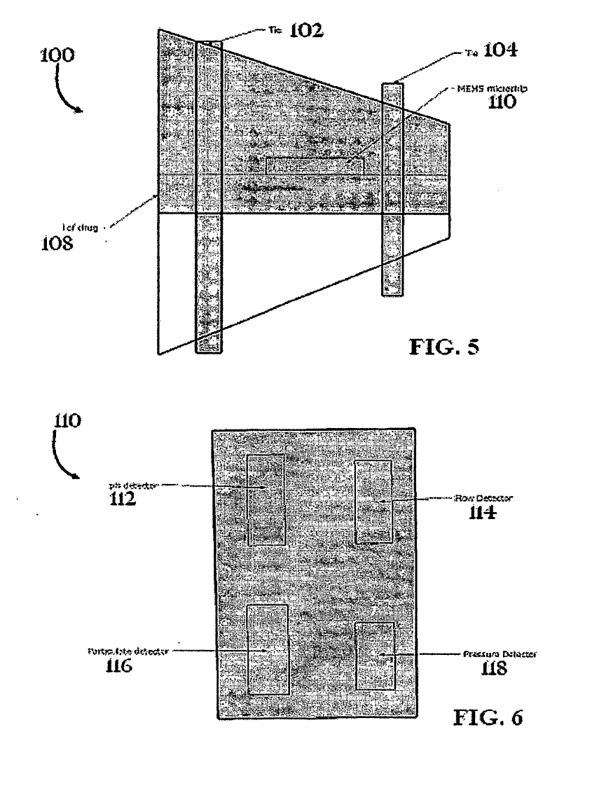

Turning next to the high-level schematic depicting a monitoring device 100 according to the invention, in FIG. 5: one can appreciate that the device may have tie-downs 102, 104 shown here, by way of example, to peripherally surround a cylindrical-cone shaped structure 100 through which a channel/fluid pathway 108 extends so that the chip-style device 110 having built-in capability (i.e. having an on-board processor, as well as at least one parameter-detection element adapted for collecting the information sought for quantification of the parameter) to ‘automatically’ monitor at least one parameter of the therapeutic agent solution as it flows through the fluid channel 108. The IC capabilities may include the capacity to monitor a multitude of parameters of the fluid as it passes through the channel.

FIG. 6 is a high-level schematic depicting a few of the many possible detection-functionalities of an IC chip-style micro-unit 110: pH detector element 112; flow detector element 114; particulate detector element 116; and pressure detector element 118. Depending upon the nature of a particular element—i.e., the parameter that element is designated to measure/monitor—proximity of the chip-style unit 110 to the solution as it flows through channel 108 may be important. For example, to measure flow optically or via pressure drop/differential, it may be preferable to have at least a portion of the tiny flow detector element in direct contact with the fluid path, and so on. In the event an obstruction occurs, a drop in flow rate to, say, ˜ zero would trigger an unintended condition sounding an alarm. Also, the device is preferably equipped with an automatic shutoff valve associated with the alarm. The capability to measure a variety of parameters provides an overall auto-monitoring capacity, as explained herein, for delivering a preselected dosage of a medicinal agent, in situ, reducing the risk of overdosing or underdosing, as well as offering a potential to minimize side effect(s) and toxicity.

Additionally it can reduce the number of necessary administrations, provide more localized and better use of the active agents, and increase patient compliance. Thus, use of the new monitoring device and associated new catheter technique, permits clinicians to deliver drugs/medicinal remedies (whether the agent requires extremely close monitoring due to a potential for toxicity, or simply are of a type typically not well-tolerated when administered systemically)—in the case of intrathecal delivery, avoiding side effects seen in IV or PO administration—directly in to the CNS, or directly into the blood stream—in the case of administering IV solutions and/or insulin therapies.

In summary fashion at a high-level, FIGS. 7-10 depict certain core, as well as additional, features of a device 100A, 100B, 320A, 320B (having selected capabilities, such as those of device 100 in FIG. 5, and elsewhere) in positional relationship within a patient body such as that respectively outlined at 200A, 200B, 300A, 300B: FIG. 7 depicts a pump unit 10A, monitoring device 100A, intrathecal catheter assembly within a patient body 200A; FIG. 8 depicts a pump unit 10B, monitoring device 100B, intrathecal catheter assembly within a patient body 200B; FIG. 9 depicts an IV (intravenous) bag unit 312A, monitoring device 320A, and IV catheter assembly in communication with the blood stream within a patient body 300A; and FIG. 10 depicts an insulin pump unit 312B, monitoring device 320B, and IV catheter assembly in communication with the blood stream within a patient body 300B. A medicine access port 88A, 88B, 388 is shown for filling pump unit 10A, 10B, 312B (whether initially, or to add constituents, modify or correct the solution as a result of information collected by the new device 100A, 100B indicating a need to do so).

FIGS. 7-10 illustrate device 100A, 100B, 320A, 320B containing an IC chip-style element respectively at 110A, 110B, 340A, 340B downstream or upstream of, and/or interconnected/retrofit with, suitable connection assembly 132/133/130A, 132/133/130B, 332/333A, 332/333B (which, in turn, may be comprised of items structured similar to those shown and labeled 32, 33, and/or 30 of FIG. 1C); likewise alternatively, the device 100A, 100B, 320A, 320B may be shaped in a manner to replace a feature similar to that at 30 in FIGS. 1D and 2. Information collected by electronic devices/ICs 110A, 110B, 340A, 340B may be transmitted from respective monitoring devices 100A, 100B, 320A, 320B in a remote/wireless manner, e.g., using RF (radio frequency) waves emitted from an ‘on-board’ wireless transmitter (which may also include receiving capability, so as to received instructions transmitted ‘remotely’ to the device).

While FIGS. 7 and 9 illustrate the new monitoring device 100A, 320A ‘external’ to the a pump unit 10A or respectively a IV bag unit 312A so that solutions passing through the device 100A, 320A have exited the pump or IV bag, the new device may be incorporated within a programmable pump assembly as suggested in FIGS. 8 and 10. As shown therein, the device 100B, 320B may be incorporated within a housing for the pump unit 10B, 312B and in communication with an exit port (such as at 132/133/130B and 332/333B) of the pump. As one can appreciate, solution flowing out unit 10A, 312A passes through the catheter (FIG. 7 referencing an intrathecal type and FIG. 8 referencing an IV type) before being directed into, respectively, a patient's CSN (210A, FIG. 8) or blood stream (310A, FIG. 10). Likewise, solution flowing out unit 10B, 312B passes through the catheter (FIG. 8 referencing an intrathecal type and FIG. 10 referencing an IV type) before being directed into, respectively, a patient's CSN (210B, FIG. 8) or blood stream (310B, FIG. 10). In any case, the handy retrofit device 100A, 100B, 320A, 320B may be accommodated for auto-monitoring of the therapeutic agent solution passing through a respective fluid channel (such as that at 108, FIG. 5) of the device.

### D. Methods of Use

In another aspect of the invention, method for automatically monitoring a plurality of parameters of a therapeutic agent solution in connection with the MPM devices described herein are also provided. The methods may generally include: (a) after having exited an automatically-driven pump mechanism, directing the therapeutic agent solution through a fluid channel of an in-line MPM device; (b) prior to being centrally administered in connection with treatment of a CNS-related condition or disorder, and while the solution flows through the fluid channel, automatically detecting at least one of the parameters using an integrated circuit (IC) unit comprising at least one parameter-detection element adapted for collecting information concerning the parameter; and (c) processing, on-board the integrated circuit (IC) unit, at least a portion of any of the information so collected.

Associated methods are contemplated for monitoring, in-line, a plurality of parameters of a therapeutic agent solution as it passes, and prior to being (a) intravenously administered to a patient in connection with a treatment, such as is the case where the therapeutic agent solution comprises an IV administerable solution, or (b) administered to a patient in connection with a treatment for diabetes, such as is the case where the therapeutic agent comprises insulin.

To assist in understanding the present invention, the following Examples are included. The experiments described herein should not, of course, be construed as specifically limiting the invention and such variations of the invention, now known or later developed, which would be within the purview of one skilled in the art are considered to fall within the scope of the invention as described herein and hereinafter claimed.

### EXAMPLES

The present invention is described in more detail with reference to the following non-limiting examples, which are offered to more fully illustrate the invention, but are not to be construed as limiting the scope thereof.

### Example 1

Operating parameters of a MPM device such as described herein, may include, by way of example only:
Pressure: 12-15 mmHg for a fluid with density like water.
Flow: 100 uL per day or 4 uL per hour, 12 cc volume in 3 months, continuous flow
Precipitation: detect particles around 10 um
pH: 6-8 no decomposition
battery: lasts for five years, supplies enough power for all sensors
alarm (internal or external): audible or vibrational, features auto shut-off that is reversible with intervention.

### Example 2

Formulations for use with a MPM device such as described herein, may include, by way of example only:

Central delivery of a psychiatric agent for treating schizophrenia—according to certain embodiments the invention—employs a reformulation of currently available clozapine in a composition that is less dense than the CSF, permitting the agent to spread rostrally. The central delivery technique according to this aspect of the invention utilizing the MPM device, permits lower doses of the antipsychotic agent to be employed. This, in turn, decreases systemic exposure to the antipsychotic agent and may decrease the patient compliance issues often currently associated with traditional (oral and IM) administration of the antipsychotic agent.

By way of example, only, in the case of the antipsychotic agent, clozapine, new formulations include stable compositions of:
a. Composition 1—Clozaril in a water soluble mixture at an adequate CSF concentration to treat symptoms; or
b. Composition 2—Recipe of Clozaril+anticonvulsant (to decrease likelihood of seizure and increasing tx of disorder)such as those distributed as Valproate, Lamictal, Oxcarbamezapine.
c. Composition 3—Recipe of Clozaril+a second antipsychotic to facilitate treatment (to decrease likelihood of seizure and increasing tx of disorder) such as those distributed as olanzapine, aripiprazole, haloperidol, etc.

While certain representative embodiments and details have been shown for the purpose of illustrating features of the invention, those skilled in the art will readily appreciate that various modifications, whether specifically or expressly identified herein, may be made to these representative embodiments without departing from the novel core teachings or scope of this technical disclosure. Accordingly, all such modifications are intended to be included within the scope of the claims.

1: A multi-parameter monitoring (MPM) device for use in-line with a central administration device to monitor a therapeutic agent solution passing therethrough prior to being centrally administered in connection with the treatment of a CNS-related condition or disorder in a subject, the MPM device comprising:
(a) a fluid channel through which the therapeutic agent solution is directed after having exited an automatically-driven pump mechanism;
(b) an integrated circuit (IC) unit comprising at least one parameter-detection element adapted for collecting information concerning at least one parameter of the therapeutic agent solution while the solution flows through said fluid channel; and
(c) on-board said integrated circuit (IC) unit is a processor adapted for processing at least a portion of any said information so collected.

2: The MPM device of claim 1, wherein the therapeutic agent solution comprises a formulation of clozapine.

3: The MPM device of claim 1, wherein the therapeutic agent solution comprises a medicine selected from the group consisting of: immunoglobulins, tegretol, lithium, felbamate, phenytoin, lamictal, phenobarbital, olanzapine, risperidone, ethosuximide, L-Dopa, pamate, phenelzine, isocarboxazid, clomipramine, bromocriptine, clozapine, progabide, oxcarbamazipine, clorazepate, etobarb, ziprasidone, seroquel, aripiprazole, zonisamide, methadone, buprinorphine, duramorph, clonidine, clonazapate, diazepam, temezapam, oxazepam, lorezapam, luvoxk paroxetine, fluoxetine, amitryptiline, nortryptiline, desipramine, amantadine, salicylic acid, ibuprofen, acetimonophen, haloperidol, loxitane, navane, mellaril, thorazine, moban, trilafon, stelazine, prolixin, prednisilone, dexamethasone, carbamezapine, valproic acid, clonezepam, ethosuximide, oxezapam, alprazolam, bromazepam, chlordiazepoxide, clobazam, clonazepam, estazolam,, flurazepam halazepam, ketazolam, quazepam, prazepam, temazepam, triazolam, nitrazepam, diamox, ACTH, carbatrol, diastat, felbamate, valproic acid, carbamezapine, lorazepam, flurazepam, clonazepam, triazolam, chlordiazepoxide, temazepam, alprazolam, sulfasalazine, acetaminophen, cafergot, and naloxone, chlorpromazine, fluphenazine, loxapine, thioridazine, thiothixine, prochlorperazine, trifluoperazine, methylprestone, lorazepam, flurazepam, clonazepam, triazolam, chlordiazepoxide, temazepam, alprazolam, hydroxyzine oxcarbazepine, zarontin, lamotrigine, aripriprazole, and olanzapine.

4: The MPM device of any of claim 1, wherein the CNS-related condition or disorder is selected from the group consisting of: schizophrenia; bipolar mood disorder; depression; shizoaffective disorder; Dementia/Alzheimer's disease; Epilepsy; Encephalitis; Multiple sclerosis; Closed head injury; Anxiety; Psychosis; Parkinson's disease; and Drug addiction.

5: The MPM device of claim 1, wherein the MPM device is releasably-connectable in-line within a central administration catheter assembly comprising a length of catheter tubing and a port-connector; said port-connector adapted for connection of said catheter assembly to an exit port of an implantable unit comprising said automatically-driven pump mechanism.

6: The MPM device of claim 5, wherein the MPM device is generally cylindrical in shape and further adapted for threaded engagement with said port-connector of said catheter assembly and for engagement with said exit port of said implantable unit.

7: The MPM device of claim 1, wherein the MPM device is integrated in-line within a central administration catheter assembly comprising a length of catheter tubing and a port-connector; said port-connector adapted for connection of said catheter assembly to an exit port of an implantable unit comprising said automatically-driven pump mechanism.

8. The MPM device of claim 1, wherein:
(a) both said automatically-driven pump mechanism, in communication with the device, are contained within a housing for an implantable unit; and
(b) the device is also in communication with an exit port of said implantable unit.

9: The MPM device of claim 8, wherein the therapeutic agent solution flows through said fluid channel prior to exiting said implantable unit through said exit port and into a port-connector of an intrathecal catheter assembly comprising a length of catheter tubing.

10: The MPM device of claim 1, wherein said parameter is selected from the group consisting of speed of flow of the therapeutic agent solution through said fluid channel; pressure within said fluid channel while the therapeutic agent solution is passing therethrough; density of the therapeutic agent solution while passing through said fluid channel; temperature within said fluid channel while the therapeutic agent solution is passing therethrough; concentration of a selected constituent of the therapeutic agent solution; concentration of a component added to the therapeutic agent solution; presence of a contaminant; concentration of a contaminant; precipitation quantity of a particle having precipitated out of the therapeutic agent solution; pH of the therapeutic agent solution; tonicity of the therapeutic agent solution; conductance of the therapeutic agent solution, and combinations thereof.

11: The MPM device-of claim 1, wherein the MPM device, at least in part, monitors for infection and/or blockage of the central administration device by collecting information concerning said at least one parameter, wherein said parameter is selected from the group consisting of: salinity, pH, particulate matter, flow, pressure, and combinations thereof.

12: The MPM device of claim 1, further comprising an alarm in communication with said processor; said alarm to activate when any said information so collected indicates a condition occurring outside a physiologic or predetermined range.

13: The MPM device of claim 12, further comprising an automatic shutoff, wherein the automatic shutoff is triggered by said alarm.

14: The MPM device of claim 1, wherein said integrated circuit (IC) unit further comprises a second parameter-detection element adapted for collecting second information concerning a second parameter of the therapeutic agent solution while the solution flows through said fluid channel; said processor further adapted for processing at least a portion of any said second information so collected.

15: The MPM device of claim 14, wherein said integrated circuit (IC) unit further comprises:
(a) a third parameter-detection element adapted for collecting third information concerning a third parameter of the therapeutic agent solution while the solution flows through said fluid channel;
(b) a fourth parameter-detection element adapted for collecting fourth information concerning a fourth parameter of the therapeutic agent solution while the solution flows through said fluid channel; and
(c) said processor further adapted for processing at least a portion of any said third information and a portion of any said fourth information so collected.

16: The MPM device of claim 15, wherein each said parameter is different from each of the other of said parameters, and is selected from the group consisting of: speed of flow of the therapeutic agent solution through said fluid channel; pressure within said fluid channel while the therapeutic agent solution is passing therethrough; density of the therapeutic agent solution while passing through said fluid channel; temperature within said fluid channel while the therapeutic agent solution is passing therethrough; concentration of a selected constituent of the therapeutic agent solution; concentration of a component added to the therapeutic agent solution; presence of a contaminant; concentration of a contaminant; precipitation quantity of a particle having precipitated out of the therapeutic agent solution; pH of the therapeutic agent solution; tonicity of the therapeutic agent solution; and conductance of the therapeutic agent solution.

17: The MPM device of claim 1, further comprising a module configured to record and/or report collected data.

18: A method for automatically monitoring a plurality of parameters of a therapeutic agent solution being centrally administered in connection with the treatment of C NS-related condition or disorder in a subject in need thereof, the method comprising:
(a) after having exited an automatically-driven pump mechanism, directing the therapeutic agent solution through the fluid channel of an in-line multi parameter monitoring (MPM) device of claim 1;
(b) prior to being centrally administered in connection with treatment of a CNS-related condition or disorder, and while the solution flows through said fluid channel, automatically detecting at least one of the parameters using the integrated circuit (IC) unit of the MPM device comprising at least one parameter-detection element adapted for collecting information concerning the parameter; and
(c) processing, on-board said integrated circuit (IC) unit, at least a portion of any said information so collected.

19: The method of claim 18, wherein the therapeutic agent solution comprises a formulation of clozapine.

20: The method of claim 18, wherein the therapeutic agent solution comprises a medicine selected from the group consisting of: immunoglobulins, tegretol, lithium, felbamate, phenytoin, lamictal, phenobarbital, olanzapine, risperidone, ethosuximide, L-Dopa, pamate, phenelzine, isocarboxazid, clomipramine, bromocriptine, clozapine, progabide, .oxcarbamazipine, clorazepate, etobarb, ziprasidone, seroquel, aripiprazole, zonisamide, methadone, buprinorphine, duramorph, clonidine, clonazapate, diazepam, temezapam, oxazepam, lorezapam, luvox, paroxetine, fluoxetine, amitryptiline, nortryptiline, desipramine, amantadine, salicylic acid, ibuprofen, acetimonophen, haloperidol, loxitane, navane, mellaril, thorazine, moban, trilafon, stelazine, prolixin, prednisilone, dexamethasone, carbamezapine, valproic acid, clonezepam, ethosuximide, oxezapam, alprazolam, bromazepam, chlordiazepoxide, clobazam, clonazepam, estazolam,, flurazepam halazepam, ketazolam, quazepam, prazepam, temazepam, triazolam, nitrazepam, diamox, ACTH, carbatrol, diastat, felbamate, valproic acid, carbamezapine, lorazepam, flurazepam, clonazepam, triazolam, chlordiazepoxide, temazepam, alprazolam, sulfasalazine, acetaminophen, cafergot, and naloxone, chlorpromazine, fluphenazine, loxapine, thioridazine, thiothixine, prochlorperazine, trifluoperazine, methylprestone, lorazepam, flurazepam, clonazepam, triazolam, chlordiazepoxide, temazepam, alprazolam, hydroxyzine oxcarbazepine, zarontin, lamotrigine, aripriprazole, and olanzapine.

21: The method of any of claim 18, wherein the CNS-related condition or disorder is selected from the group consisting of: schizophrenia; bipolar mood disorder; depression; shizoaffective disorder; Dementia/Alzheimer's disease; Epilepsy; Encephalitis; Multiple sclerosis; Closed head injury; Anxiety; Psychosis; Parkinson's disease; and Drug addiction.

22: The method of claim 18, wherein said parameter is selected from the group consisting of: speed of flow of the therapeutic agent solution through said fluid channel; pressure within said fluid channel while the therapeutic agent solution is passing therethrough; density of the therapeutic agent solution while passing through said fluid channel; temperature within said fluid channel while the therapeutic agent solution is passing therethrough; concentration of a selected constituent of the therapeutic agent solution; concentration of a component added to the therapeutic agent solution; presence of a contaminant; concentration of a contaminant; precipitation quantity of a particle having precipitated out of the therapeutic agent solution; pH of the therapeutic agent solution; tonicity of the therapeutic agent solution; and conductance of the therapeutic agent solution.

23: A multi-parameter monitoring (MPM) device for in-line use to monitor a therapeutic agent solution passing therethrough prior to being intravenously administered to a subject in connection with a treatment, the device comprising:
(a) a fluid channel through which the therapeutic agent solution is directed after having exited an IV receptacle unit;
(b) an integrated circuit (IC) unit comprising at least one parameter detection element adapted for collecting information concerning a parameter of the J therapeutic agent solution while the solution flows through said fluid channel; and
(c) on-board said integrated circuit (IC) unit is a processor adapted for processing at least a portion of any said information so collected.

24: A multi-parameter monitoring (MPM) device for in-line use to monitor a therapeutic agent solution passing therethrough prior to being administered to a subject in connection with a treatment for diabetes, the device comprising:
(a) a fluid channel through which the therapeutic agent solution is directed after having exited an automatically-driven pump mechanism;
(b) an integrated circuit (IC) unit comprising at least one parameter-detection element adapted for collecting information concerning a parameter of the therapeutic agent solution while the solution flows through said fluid channel; and
(c) on-board said integrated circuit (IC) unit is a processor adapted for processing at least a portion of any said information so collected.
